(19) United States
(12) Patent Application Publication
(10) Pub. No.: US 2003/0050620 A1
(43) Pub. Date: Mar. 13, 2003

(54) Combinatorial type controlled release drug delivery device

(76) Inventors: Isa Odidi, Toronto (CA); Amina Odidi, Toronto (CA)

Correspondence Address: Dr. Lola A. Bartoszewicz, Sim & McBurney, 6th Floor, 330 University Avenue, Toronto, ON, M5G 1R7, CA

(21) Appl. No.: 09/947,464

(22) Filed: Sep. 7, 2001

(51) Int. Cl. A61K 9/22

(52) U.S. Cl. 604/890.1

(57) ABSTRACT

The present invention is a controlled release delivery device. The device comprises more than one vehicle comprising up to 60% by wgt active agent; up to 60% by wgt amino acid; up to 60% by wgt buffer, and up to 70% by wgt polymer. The vehicle(s) are provided within a housing.

### FIELD OF THE INVENTION

The present invention is directed to a controlled release delivery system, and more specifically to a device for the simultaneous delivery of a variety of different pharmaceutically active agents.

### BACKGROUND OF THE INVENTION

Drug delivery devices are known and used to control the release of pharmaceutically active substances. These devices operate successfully for their intended use. However, these devices are often limited in their use to deliver more than one pharmaceutically active agent concurrently. These devices are also limited in their ability to deliver pharmaceutical active substances for chronotherapeutic application, over an extended period of time or in a pulsatile manner.

It will be appreciated by those versed in the art, that if a device can be provided that allows the delivery of more than one pharmaceutically active substance concurrently, especially those that are incompatible, in a pulsatile manner for the pharmaceutically active substance, and also allow for chronotherapeutic application such a device would have a positive value and represent an advancement in the science of controlled delivery technology.

### SUMMARY OF THE INVENTION

The Applicants have developed controlled release delivery device that comprises a variety of different vehicles for delivering a variety of different pharmaceutical active agents concurrently in one simple oral dose. The delivery device is made of a combination of a variety of vehicles which comprise a population of granules, beads, pellets or tablets within a housing where each population of vehicle may contain a different combination of active agent, release modulating/controlling polymer/s, optionally nonpolar, polar/basic, polar/neutral, or polar/acidic amino acids and optionally one or more organic or inorganic buffers in an intimate physical or chemical homogeneous mixture.

This delivery system can be adapted to deliver a variety of active agents in mechanical, chemical, physical, fluid, gaseous, mobile, biological, agricultural, terrestrial, extra terrestrial, gravitational and zero gravity environments. Such adaptations are not limited in size, shape, topography, structure and composition.

It is an aspect of the invention to provide a controlled release delivery device for the controlled delivery of a pharmaceutically active agent which represents a substantial improvement and advancement in controlled drug delivery technology.

It is another aspect of the present invention to provide a controlled release delivery device that is useful for simultaneously delivering more than done pharmaceutically active substance in an orally administrable manner.

It is yet another aspect of the present invention to provide a controlled release delivery system capable of the pulsatile delivery of pharmaceutically active substances.

It is still a further aspect of the present invention to provide a controlled release delivery device that is useful for delivering pharmaceutically active substances that are typically incompatible with each other.

Yet another aspect of the present invention is to provide a controlled release delivery device comprising;

more than one vehicle comprising an active agent, an amino acid, a buffer and a polymer;

wherein said vehicle is provided within a housing.

The vehicle may additionally comprise activated or super activated charcoal.

According to an aspect of the present invention is a controlled release delivery device comprising;

more than one vehicle comprising up to 60% by wgt active agent; up to 60% by wgt amino acid; up to 60% by wgt buffer; and up to 70% by wgt polymer; wherein said vehicle is provided within a housing.

Still another aspect of the present invention is to provide a controlled drug release modulating device for chronotherapeutic application.

Still a further aspect of the present invention is to provide a controlled release modulating device comprising one or more different vehicles comprising granules, beads, pellets or tablets wherein each vehicle comprises different pharmaceutical active and different release properties and wherein one or more of the vehicles may be completely or partially coated with a polymeric coating.

Yet still a further aspect of the present invention is to provide a controlled release modulating delivery system that can be adapted to deliver one or more pharmaceutically active substances in a controlled and/or pulsatile manner and/or continuous rate over a prolonged period of time.

These together with other aspects and advantages which will be subsequently apparent, reside in the details of construction and operation as more fully hereinafter described and claimed, reference being had to the accompanying drawing forming a part hereof.

### BRIEF DESCRIPTION OF THE DRAWINGS

The device will be further illustrated by the following description of an embodiment thereof, given by way of example only with reference to the accompanying drawings in which.

FIG. 1 is a schematic drawing showing an assembly of six populations of tablets in a holding chamber/encapsulant.

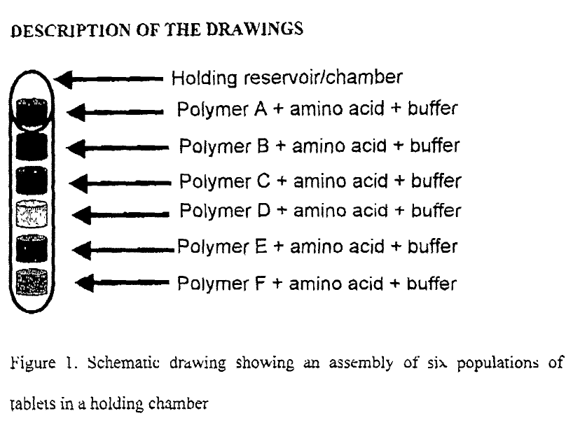

FIG. 2 is a schematic drawing showing an assembly of two populations of beads and two populations of tablets in a holding chamber/encapsulant.

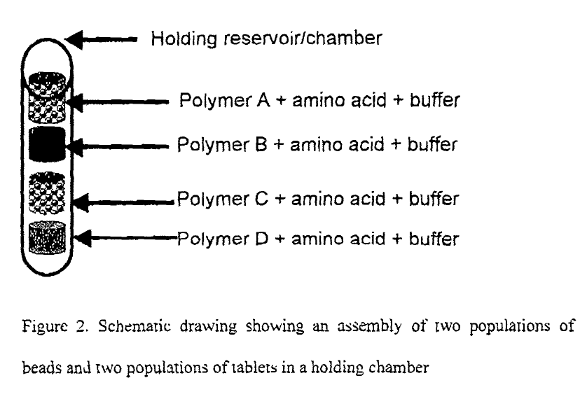

### DETAILED DESCRIPTION OF THE PREFERRED EMBODIMENTS

The invention comprises a variety of compositions contained within a housing such as a chamber or reservoir. Each population of composition contained within the housing can be made into a variety of different vehicles such as granules, beads, pellets, or tablets. Each different type of vehicle in one aspect contains a polymer, an active agent, an amino acid and a suitable buffer and then a variety of the different types of vehicles are packed into a housing. The vehicles may also additionally comprise activated or super activated charcoal.

The vehicles as made into granules, beads, pellets or tablets can be further fabricated to be regular or irregular in shape and preferably have a diameter and thickness of up to about 40 mm, and preferably up to about 20 mm and most preferably up to about 13 mm.

The polymer(s) for use in making the different vehicles of pharmaceutical formulations may be selected from the group consisting of cellulose esters, cellulose ethers, polyethylene oxide, carbomer, cyclodextrins, polyethelene glycol, dextran, polyvinylpyrrolidone, lactide/glycolide copolymers, poly(ortho esters), polyanhydrides, polyvinyl alcohol, alginates, polysaccharides, polyamides, polyvinyl chloride, polyethylene vinyl acetate, polvinyl pyrrolidone, polyurethanes, hydrogels, silicone polymers, polyacrylates, polymethacrylates, polyanhydrides, poly amino carbonates, deacetylated chitin, collagen, polyisobutylenes, gelucire, glyceryl behenate and mixtures thereof

The amino acids that may be formulated into the compositions of the invention may be selected from nonpolar, polar/basic, polar/neutral or polar/acidic amino acids and mixtures thereof.

The buffers for use in the compositions of the invention may be selected from inorganic or organic buffers such as phosphate, citrate, HEPES, succinate, histidine, maleate, lactate, and acetate buffers and mixtures thereof.

Each composition may be formulated into a variety of different vehicles such as for example granules, beads, pellets, or tablets which may also contain surfactants, cryoprotectants, lyoprotectants, excipients and mixtures thereof in amounts that are readily determined by one of skill in the art. Each vehicle whether in the form of a granule, bead, pellet or tablet may optionally be completely or partially coated with a polymeric coating.

The vehicle(s) of the invention may include a variety of active agents such as for example pharmaceuticals, chemicals, biologicals, pesticides, insecticides, algicides, fungicides, germicides and herbicides.

In one preferred aspect, the active agent comprises Acetaminophen/Codeine, Albuterol, Alendronate, Allopurinol, Alprazolam, Amtriptyline, Amlodipine, Amlodipine/Benazepril, Amoxicillin, Amoxicillin/Clavulanate, Amphetamine Mixed Calsts, acarbose, Atelolol, Atorvastatin, Azithromycin, Beclomethasone, Benazepril, Bisoprolol/HCTZ, Brimonidine, Calcitonin Salmon, Carbamazepine, Carisoprodol, Carvedilol, cefprozil, Cefuroxime, Clecoxib, Cephalexin, Cetinzine, Ciprofloxacin, Cisapride, Citalopram, Clarithromycin, Clonazepam, Klondike, Clopidogrel, Clotrimazole/Betamethasone, Cyclobenzaprine, Diazepam, Misoprostol, Digoxin, Divalproex, Donepezil, Doxazosin, Enalapril, Erythromycin, Estradiol, Ethinyl Estradiol/Norethindrone, Famotidine, Felodipine, Fexofenadine, Fexofenadine/Pseudoephedrine, Fluoxetine, Fluticasone Propionate, Fluvastatin, Fluvoxamine maleate, Fosinopril, Furosemide, Gemfibrozil, Glimepiride, Glyburide, Guaifenesin/Phenylpropanolamine, Granisetron HCl, Hydrochlorothiazide, Hydrocodone w/APAP, Ibuprofen, Ipratropium, Ipratropium/Albuterol, Irbesartan, Isosorbide Mononitrate, Lansoprazole, Latanoprost, Levofloxacin, Levonorgestrel/Ethinyl Estradiol, Levothyroxine, Lisinopril, Lisinopril/HCTZ, Loratadine, Loratidine/Pseudoephedrine, Lorazepam, Losartan, Losartan/HCTZ, Lovastatin, Methylprednisolone, Methylphenidate, Metoprolol, mightol Mometasone, Montelukast, Mupirocin, Naproxen, Nitrofurantoin, Nizatidine, Olanzapine, Oxaprozin, Oxycodone, Oxycodone/APAP, Paroxetine, Penicillin VK, Phenytoin, Potassium, Chloride, Pramipexole HCl, Pravastatin, Predinisone, Promethazine, Propoxyphene N/APAP, Propranolol, Quinapril, Raloxifene, Ramipril, Ranitidine, repaglinide, Risperidone, Rofecoxib, Salmeterol, Sertraline, Sildenafil Citrate, Simvastatin, Sumatriptan, Tamoxifen, Tamsulosin, Tamazepam, Terazosin, Terbinafine, Tobramycin/Dexamethasone, Tolterodine, Tranylcypromine sulfte, Trazodone, Triamterene/HCTZ, Troglitazone, Valsartin, Venlafaxin, Warfarin, Zafirlukast and Zolpidem.

In a further embodiment the active agent comprises one or more of the drugs used in HIV or AIDS treatment such as for example Abacavir, amprenavir, stavudine, zalcitabine, didanosine, delavirdine, efavirenz Hydroxyurea, indinavir, lamivudine, lopinavir, nelfinavir, nevirapine, ritonavir Saquinavir, stavudine and zidovudine.

In still another embodiment, the active agent comprises one or more proteins, peptides, hormones, prostaglandins, and anticancer agents.

In yet a further embodiment, the active agent comprises active or inactive metabolites of active pharmaceutical agents ingredients or salts of the metabolites.

The active or inactive metabolites of active pharmaceutical ingredients or salts of the metabolites may be administered systemically to humans or animals by way of incorporating the active pharmaceutical ingredient as prodrug which on administration generates the active or inactive metabolites.

The combinatorial type of controlled release delivery device in accordance with the present invention may be manufactured using conventional granulation, peletization, tabletting and/or coating technologies. As an example, a homogeneous blend of the pharmaceutically active substance, polymer, amino acid, buffer, surfactant, cryoprotectant and lyoprotectant are granulated, dried and milled. Alternatively, the homogeneous blend is granulated, extruded and dried The resulting dried granules are lubricated and compressed into a preselect shape to form one population of a selected type of vehicle. Other populations of vehicles are similarly manufactured except that is it preferred that a different polymer is used each time for each type of vehicle.

A complete or partial coating may be applied on one or more of the vehicle populations by spraying, molding and/or dipping. Finally, the various population of vehicles in the from of granules, beads, pellets and tablets are assembled in no particular order within a housing such as a chamber chamber/reservoir.

In accordance with the present invention the housing that forms the chamber or reservoir for encapsulating the various vehicles comprising the different pharmaceutical formulations therein may additionally contain a non-toxic metal or metal alloy such as for example titanium, platinum and gold The housing may also contain non-toxic plastic, hard gelatin or hydroxypropyl methyl cellulose.

The device of the invention is suitable for oral ingestion as well as via sublingual, intraocular, intramuscular, subcutaneous, anal and vaginal use as well as for implantation to a desired location within the body. The device of the present invention can be used for a variety of different applications including for human and veterinary use and agricultural use.

The controlled release drug delivery system as taught in the preset invention provides a novel device in which a housing has incorporated therein a variety of different compositions in the form of pellets, granules, beads and tablets that each may provide a different form of extended release used for the unexpected and unobvious but precise delivery of similar, dissimilar or incompatible substances at controlled rates in a pulsatile and/or prolonged manner in the environment of use or for chronotherapeutic application.

### EXAMPLES

These examples should not be construed to be limiting in scope, they are merely illustrative of the present invention. These and other examples will become apparent to those versed in the art in the light of the present disclosure, the drawings and the claims contained therein.

### Example 1

A combinatorial type controlled release modulator comprising a housing containing four populations of vehicles as tablets was manufactured as follows:

Composition, Manufacture and Assembly of Tablets:

| | Tablet 1 | Tablet 2 | Tablet 3 | Tablet 4 |
|---|---|---|---|---|
| | (mg) | (mg) | (mg) | (mg) |
| Nisoldipine | 10 | 10 | 10 | 10 |
| Hydroxypropyl methyl cellulose | 15 | — | — | — |
| Xanthan gum | — | 10 | — | — |
| Polvinyl acetate/Polyvinyl | — | — | 10 | — |
| pyrrolidone (PVA/PVP copolymer) | | | | |
| Glyceryl behenate | — | — | — | 5 |
| Lactose | 45 | 50.5 | 51 | 56 |
| Silicone dioxide | 1 | 1 | 1 | 1 |
| Arginine | 10 | 10 | 10 | 10 |
| Microcrystaline cellulose | 12 | 22 | 12 | 12 |
| Sodium phosphate | 1 | 1 | 1 | 1 |
| Sodium lauryl sulphate | 3 | 2.5 | 2 | 2 |
| Magnesium stearate | 1 | 1 | 1 | 1 |

Each tablet population was manufactured by wet granulation of a homogeneous blend of the pharmaceutically active substance, polymer, amino acid, buffer, surfactant, cryoprotectant, lyoprotectant, and pharmaceutical excipients. The wet granules were dried and milled. The resulting milled granules were lubricated and compressed into a preselected shape to form one population. A partial coat of pH reactive coating was applied onto the tablet population designated Tablet 1 above by coating in a perforated side vented coating pan. Finally, one tablet from each of the four population of tablets described above were assembled in no particular order in a housing made of hard gelatin or hydroxypropyl methyl cellulose.

### Example 2

A combinatorial type controlled release device comprising a housing containing two population of tablets was manufactured as follows:

Composition, Manufacture and Assembly of Tablets:

| | Tablet 1 | Tablet 2 |
|---|---|---|
| | (mg) | (mg) |
| Felodipine | 5 | 5 |
| Hydroxypropyl methyl cellulose | 10 | — |
| Glyceryl behenate | — | 5 |
| Lactose | 71 | 67 |
| Silicone dioxide | 1 | 1 |
| Arginine | 5 | 5 |
| Microcrystaline cellulose | 12 | 12 |
| Sodium phosphate | 1 | 1 |
| Sodium lauryl sulphate | 3 | 3 |
| Magnesium stearate | 1 | 1 |

Each tablet population was manufactured by wet granulation of a homogeneous blend of the pharmaceutically active substance, polymer, amino acid, buffer, surfactant, cryoprotectant, lyoprotectant and pharmaceutical excipients. The wet granules were dried and milled. The resulting milled granules were lubricated and compressed into a preselected shape to form one population. A complete coat of pH reactive coating was applied onto the tablet population designated Tablet 2 above by coating in a perforated side vented coating pan. Finally, one tablet from each of the two population of tablets were assembled in no particular order in a housing made of hard gelatin or hydroxypropyl methyl cellulose.

### Example 3

A combinatorial type controlled release delivery device comprising a housing containing three population of tablets was manufactured as follows:

Composition, Manufacture and Assembly of Tablets:

| | Tablet 1 | Tablet 2 | Tablet 3 |
|---|---|---|---|
| | (mg) | (mg) | (mg) |
| Losartan potassium | 25 | — | 25 |
| Hydrochlorothiazide | — | 12.5 | |
| Hydroxypropyl methyl cellulose | 15 | — | |
| Compitrol | — | — | 10 |
| Lactose | 47 | 58.5 | 42 |
| Silicone dioxide | 1 | 1 | 1 |
| Crospovidone | — | 5 | — |
| Arginine | 5 | 5 | 5 |
| Microcrystaline cellulose | 15 | 17 | 15 |
| Sodium phosphate | 1 | 1 | 1 |
| Magnesium stearate | 1 | 1 | 1 |

Each tablet population was manufactured by wet granulation of a homogeneous blend of the pharmaceutically active substance, polymer, amino acid, buffer, cryoprotectant and pharmaceutical excipients. The wet granules were dried and milled. The resulting milled granules were lubricated and compressed into a preselected shape to form one population. A complete coat of pH reactive coating was applied onto the tablet population designated Tablet 3 above by coating in a perforated side vented coating pan. Finally, one tablet from each of the two population of tablets were assembled in no particular order in the holding chamber/reservoir made of hard gelatin or hydroxypropyl methyl cellulose.

### Example 4

A combinatorial type controlled release modulator, comprising a holding chamber containing four populations of tablets was manufactured as follows:

Composition, Manufacture and Assembly of Tablets:

| | Tablet 1 | Tablet 2 | Tablet 3 | Tablet 4 |
|---|---|---|---|---|
| | (%) | (%) | (%) | (%) |
| Dextroamphetamine | 1.25-10 | — | — | — |
| Saccharate | | | | |
| Amphetamine | — | 1.25-10 | — | — |
| Aspartate | | | | |
| Dextroamphetamine | — | — | 1.25-10 | — |
| Sulfats USP | | | | |
| Amphetamine Sulfact | — | — | — | 1.25-10 |
| USP | | | | |
| Hydroxypropyl methyl | 5-25 | — | — | — |
| cellulose | | | | |
| Hydroxypropyl | — | 5-25 | — | — |
| Cellulose | | | | |
| Polyvinyl acetate/ | — | — | 5-25 | — |
| Polyvinyl pyrrolidone | | | | |
| (PVA/PVP copolymer) | | | | |
| Glyceryl behenate | — | — | — | 5-25 |
| Lactose | 47.75-57.75 | 50.5 | 51 | 56 |
| Silicone dioxide | 1 | 1 | 1 | 1 |
| Arginine | 5 | 5 | 5 | 5 |
| Microcrystaline | 181 | 17 | 15 | 17 |
| cellulose | | | | |
| Sodium phosphate | 1 | 1 | 1 | 1 |
| Sodium lauryl sulphate | 1 | 1 | 1 | 1 |
| Magnesium stearate | 1 | 1 | 1 | 1 |

Each tablet population was manufactured by wet granulation of a homogeneous blend of the pharmaceutically active substance, polymer, amino acid, buffer, surfactant, cryoprotectant, lyoprotectant and pharmaceutical excipients. The wet granules were dried and milled. The resulting milled granules were lubricated and compressed into a preselected shape to form one population. Finally, one tablet each from the four population of tablets were assembled in no particular order in a housing made of hard gelatin or hydroxypropyl methyl cellulose.

### Example 5

A combinatorial type controlled release modulator, comprising a holding chamber containing four population of beads was manufactured as follows:

Composition, Manufacture and Assembly of Beads:

| | Bead 1 | Bead 2 | Bead 3 | Bead 4 |
|---|---|---|---|---|
| | (%) | (%) | (%) | (%) |
| Carvedilol | 3.125 | 3.125 | 3.125 | 3.125 |
| Hydroxypropyl methyl cellulose | 5-25 | — | — | — |
| Hydroxyethyl Cellulose | — | 5-25 | — | — |
| Polyvinyl acetate/ | — | — | 5-25 | — |
| Polyvinyl pyrrolidone | | | | |
| Ethycellulose | — | — | — | 5-25 |
| Silicone dioxide | 1 | 1 | 1 | 1 |
| Arginine | 2 | 2 | 2 | 2 |
| Microcrystaline cellulose | 70 | 70 | 70 | 70 |
| Sodium phosphate | 1 | 1 | 1 | 1 |

Each bead population was manufactured by wet massing of a homogeneous blend of the pharmaceutically active substance, polymer, amino acid, buffer, cryoprotectant, lyoprotectant and pharmaceutical excipients. The wet mass was extruded and the extrudate spheronized. The resulting spheronoids were dried in a conventional oven. A complete coat of pH reactive coating was applied onto beat population designated Bead 2 and Bead 3 above by coating in a fluid, bead coater. Finally, 100 mg each from the different population of heads were assembled in no particular order in the holding chamber/reservoir made of hard gelatin or hydroxypropyl methyl cellulose.

### Example 6

Same as in example 5, except that the different population of beads were coated with a pH independent coating such as non pH reactive methacrylic acid copolymer.

### Example 7

Same as in example 5, except that the bead population designated Bead 1 and Bead 3 were coated with a pH reactive coating while bead population designated Bead 2 and Bead 4 were coated with a pH independent coating such as a non pH reactive methacrylic acid copolymer.

### Example 8

A combinatorial type controlled release modulator, comprising a holding chamber containing three population of tablets was manufactured as follows:

Composition, Manufacture and Assembly of Tablets:

| | Tablet 1 | Tablet 2 | Tablet 3 |
|---|---|---|---|
| | (mg) | (mg) | (mg) |
| Carbamazepine | 100 | 100 | 100 |
| Hydroxyethyl Cellulose | 60 | — | — |
| Hydroxypropyl methyl cellulose | — | 60 | — |
| Xanthan Gum | — | — | 32 |
| Silicone dioxide | 1 | 1 | 1 |
| Activated or super activated charcoal | 3 | 3 | 3 |
| Lactose | 33 | 33 | 33 |
| Sodium lauryl sulphate | 5 | 5 | 5 |
| Xanthan Gum | — | — | 32 |
| Arginine | 5 | 5 | 5 |
| Microcrystaline cellulose | 15 | 15 | 15 |
| Citric acid | 1 | 1 | 1 |
| Magnesium stearate | 2 | 1 | 1 |

Each tablet population was manufactured by wet granulation of a homogeneous blend of the pharmaceutically active substance, polymer, amino acid, buffer, cryoprotectant and pharmaceutical necessities. The wet granules are dried and milled. The resulting milled granules are lubricated and compressed into a preselected shape to form one population Finally, one tablet each form the three population of tablets are assembled in no particular order in the holding chamber/reservoir made of hard gelatin or hydroxypropyl methyl cellulose.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

What is claimed is:

1. A controlled release delivery device comprising;
more than one vehicle comprising up to 60% by wgt active agent; up to 60% by wgt amino acid; up to 60% by wgt buffer, and up to 70% by wgt polymer; wherein said vehicle is provided within a housing.

2. The device of claim 1, wherein said vehicle is provided as granules, beads, pellets or tablets.

3. The device of claim 2, wherein said amino acid is selected from the group consisting of nonpolar, polar neutral, polar basic and polar/acid amino acids.

4. The device of claim 2, wherein the buffer is selected from the group consisting of organic and inorganic buffers.

5. The device of claim 4, wherein said buffer is selected from the group consisting of phosphate, citrate, HEPES, succinate, histidine, maleate, lactate, and acetate buffers and mixtures thereof.

6. The device of claim 2, wherein said polymer is selected from the group consisting of cellulose esters, cellulose ethers, polyethylene oxide, carbomer, cyclodextrins, polyethelene glycol, dextran, polyvinylpyrrolidone, lactide/glycolide copolymers, poly(ortho esters), polyanhydrides, polyvinyl alcohol, alginates, polysaccharides, polyamides, polyvinyl chloride, polyethylene vinyl acetate, polvinyl pyrrolidone, polyurethanes, hydrogels, silicone polymers, polyacrylates, polymethacrylates, polyanhydrides, poly amino carbonates, deacetylated chitin, collagen, polyisobutylenes, gelucire, glyceryl behenate and mixtures thereof.

7. The device of claim 1, wherein said housing is made of a material selected from the group consisting of gelatin, hydroxypropyl methyl cellulose, a non-toxic metal, or metal allow and a non-toxic plastic or a combination thereof.

8. The device of claim 2, wherein the granules, pellets, beads or tablets may be regular or irregular in shape.

9. The device of claim 2, wherein the granules, pellets, beads or tablets have a diameter and thickness of less than about 40 mm.

10. The device of claim 9, wherein the granules, pellets, beads or tablets have a diameter and thickness of less than about 13 mm.

11. The device of claim 2, wherein said vehicle additionally comprises an agent selected from the group consisting of cryoprotectant, lyoprotectant and surfactant.

12. The device of claim 2, wherein said device additionally comprises activated or super activated charcoal.

13. The device of claim 1, wherein said active agent is selected from the group consisting of a pharmaceutical active, protein, peptide, algicide, fungicide, germicide, herbicide, insecticide, pesticide and mixtures thereof.

14. The device of claim 13, wherein said pharmaceutical active is selected from the group consisting of Acetaminophen/Codeine, Albuterol, Alendronate, Allopurinol, Alprazolam, Amitripryline, Amlodipine, Amlodipine/Benazepril, Amoxicillin, Amoxicillin/Clavulanate, Amphetamine Mixed Calsts, acarbose, Atelolol, Atorvastatin, Azithromycin, Beclomethasone, Benazepril, Bisoprolol/HCTZ, Brimonidine, Calcitonin Salmon, Carbamazepine, Carisoprodol, Carvedilol, cefprozil, Cefuroxime, Clecoxib, Cephalexin, Cetinzine, Ciprofloxacin, Cisapride, Citalopram, Clarithromycin, Clonazepam, Clonidine, Clopidogrel, Clotrimazole/Betamethasone, Cyclobenzaprine, Diazepam, Misoprostol, Digoxin, Divalproex, Donepezil, Doxazosin, Enalapril, Erythromycin, Estradiol, Ethinyl Estradiol/Norethindrone, Famotidine, Felodipine, Fexofenadine, Fexofenadine/Pseudoephedrine, Fluoxetine, Fluticasone Propionate, Fluvastatin, Fluvoxamine maleate, Fosinopril, Furosemide, Gemfibrozil, Glimepiride, Glyburide, Guaifenesin/Phenylpropanolamine, Granisetron HCl, Hydrochlorothiazide, Hydrocodone w/APAP, Ibuprofen, Ipratropium, Ipratropium/Albuterol, Irbesartan, Isosorbide Mononitrate, Lansoprazole, Latanoprost, Levofloxacin, Levonorgestrel/Ethinyl Estradiol, Levothyroxine, Lisinopril, Lisinopril/HCTZ, Loratadine, Loratidine/Pseudoephedrine, Lorazepam, Losartan, Losartan/HCTZ, Lovastatin, Methylprednisolone, Methylphenidate, Metoprolol, miglitol Mometasone, Montelukast, Mupirocin, Naproxen, Nitrofurantoin, Nizatidine, Olanzapine, Oxaprozin, Oxycodone, Oxycodone/APAP, Paroxetine, Penicillin VK, Phenytoin, Potassium, Chloride, Pramipexole HCl, Pravastatin, Predinisone, Promethazine, Propoxyphene N/APAP, Propranolol, Quinapril, Raloxifene, Ramipril, Ranitidine, repaglinide, Risperidone, Rofecoxib, Salmeterol, Sertraline, Sildenafil Citrate, Simvastatin, Sumatriptan, Tamoxifen, Tamsulosin, Tamazepam, Terazosin, Terbinafine, Tobramycin/Dexamethasone, Tolterodine, Tranylcypromine sulfte, Trazodone, Triamterene/HCTZ, Troglitazone, Valsartin, Venlafaxin, Warfarin, Zafirlukast and Zolpidem.

15. The device of claim 13, wherein said active agent is one to treat HIV or AIDS and is selected from the group consisting of Abacavir, amprenavir, stavudine, zalcitabine, didanosine, delavirdine, efavirenz Hydroxyurea, indinavir lamivudine, lopinavir, nelfinavir, nevirapine, ritonavir Saquinavir, stavudine and zidovudine.

16. The device of claim 13, wherein said pharmaceutical active is selected from the group consisting of hormones and prostaglandins.

17. The device of claim 13, wherein said pharmaceutical active is an anticancer agent.

18. The device of claim 13, wherein said active agent is an active or inactive metabolite or salt thereof, of a pharmaceutical agent.

19. The device of claim 13, wherein two or more vehicles are provided wherein at least one vehicle provides a zero order release and at least one vehicle provides a first order release of pharmaceutically active substance.

20. The device of claim 13, wherein at least one vehicle provides a zero order release of pharmaceutically active substance.

21. The device of claim 13, wherein at least one vehicle provides a first order release of pharmaceutically active substance.

22. The device of claim 13, wherein at least one vehicle provides a pseudo first order release of pharmaceutically active substance

23. The device of claim 13, wherein said device provides for the controlled release delivery of more than one pharmaceutically active substance that are incompatible.

24. A controlled release delivery device comprising:
more than one vehicle comprising up to 60% by wgt active agent; up to 60% by wgt amino acid; up to 60% by weight buffer; up to 70% by wgt polymer, one or more agents selected from the group consisting of cryoprotectant, lyoprotectant, surfactant, activated charcoal and super activated charcoal;
wherein said vehicle is provided within a housing made or a material selected from the group consisting of gelatin, hydroxypropyl methyl cellulose, non-toxic metal or metal alloy and non-toxic plastic.
